(19) United States
(12) Patent Application Publication
(10) Pub. No.: US 2010/0204438 A1
(43) Pub. Date: Aug. 12, 2010

(54) METHOD FOR PRODUCING TEDA DERIVATIVES

(75) Inventors: Gunther Van Cauwenberge, Temse (BE); Johann-Peter Melder, Bohl-Iggelheim (DE); Joachim-Thierry Anders, Gonnheim (DE); Christoph Benisch, Mannheim (DE); Rainer Klopsch, Worms (DE); Gregor Daun, Neckargemund (DE); Christian Dully, Ludwigshafen (DE); Boris Buschhaus, Mannheim (DE); Henning Böckemeier, Gottingen (DE); Evelyn Pox, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/676,254

(22) PCT Filed: Aug. 29, 2008
(86) PCT No.: PCT/EP08/61393

§ 371 Date: Mar. 3, 2010

(30) Foreign Application Priority Data
Sep. 3, 2007 (EP) 07115513.9

(51) Int. Cl. C08G 73/00; C07D 487/00

(52) U.S. Cl. 528/423; 544/352; 544/351

(57) ABSTRACT

The present invention relates to a process for preparing triethylenediamine (TEDA) derivatives, comprising the following steps:
a) reacting a dihydropyrazine with an olefin,
b) if appropriate hydrogenating after step a).

The present invention further also relates to novel TEDA derivatives as such and to their use as incorporable polyurethane catalysts.

The present invention relates to a process for preparing triethylenediamine (TEDA) derivatives proceeding from dihydropyrazines and olefins. Illustrative TEDA derivatives which can be prepared by the process according to the invention correspond to the general formula (Ia) or (Ib)

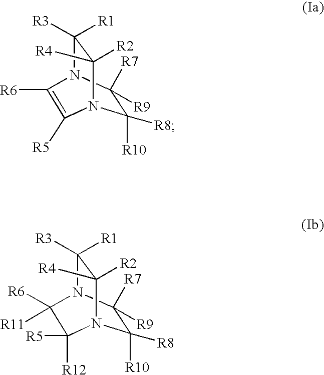

where the substituents R1 to R12 are defined in the text below. The present invention further also relates to a selection of novel TEDA derivatives of the above-defined general formula (Ia) or (Ib) and to their use as polyurethane catalysts, preferably as incorporable polyurethane catalysts.

Processes for preparing TEDA (triethylenediamine, also known as 1,4-diazabicyclo[2.2.2]octane (DABCO)) or derivatives of TEDA are known. For example, WO 01/02404 describes a process for preparing TEDA using zeolite catalysts. The reactants used in TEDA preparation are ethylenediamine (EDA) and piperazine (PIP). TEDA is an important chemical commodity whose uses include the preparation of pharmaceuticals and polymers, especially as a catalyst in the preparation of polyurethanes (PU).

An alternative process for preparing TEDA or TEDA derivatives is described in U.S. Pat. No. 6,147,185. This process relates especially to the preparation of TEDA derivatives to which one or two hydrocarbon-containing rings are fused. For example, 6-methylquinoxaline is first hydrogenated to 6-methyldecahydroquinoxaline, which then reacts with ethylene oxide. The resulting intermediate, which has a hydroxyethyl substituent on a ring nitrogen atom, is finally converted in the presence of a phosphate catalyst at 340° C. with ring closure to give the corresponding TEDA derivative (here, 6-methylcyclohexodiazabicyclo-2.2.2-octane). The compounds described in U.S. Pat. No. 6,147,185 are likewise suitable as PU catalysts.

Especially the synthesis of TEDA derivatives which have functional groups (for example an ester, ether or amino function) is found to be difficult with the above-described processes. Only a few such TEDA derivatives with a functional group are known to date. The use of such substituted TEDA derivatives as a PU catalyst is to date yet to be described.

Further TEDA derivatives are described, for example, by T. Oishi et al., Tetrahedron Letters, volume 33 (5), pages 639 to 642 (1992). Proceeding from the corresponding piperazine derivative, the corresponding TEDA derivatives which have two benzoxymethyl substituents in positions 2 and 3 of TEDA are synthesized by reaction with 1,2-dibromoethane in ethanol. Alternatively, the corresponding substituents may also have t-butyldimethylsilyl or t-butyldiphenylsilyl-containing ether substituents instead of benzyl.

WO 98/24790 relates to the preparation of TEDA derivatives, in which TEDA is linked to a benzene fraction via a methyl ester bridge. The compounds, which are used as medicaments, are prepared by firstly reacting piperazine with ethyl 2,3-dibromopropionate to give a TEDA derivative which has an ethyl ester function in the 2 position. This TEDA derivative is subsequently reduced with lithium aluminum hydride to 2-hydroxymethyl-TEDA which is subsequently reacted with the appropriate benzoic acid derivative to give the target molecule.

In L. Street et al., J. Med. Chem., 33 (1990), pages 2690 to 2697, a 2-ethylcarboxyl-substituted TEDA is prepared analogously to WO 98/24790 and then converted with ring closure to the corresponding 1,2,4-oxadiazole-TEDA derivative.

G. Shishkin et al., Chem. Heterocycl. Com., 1980, pages 1069 to 1072 describe the reduction of TEDA derivatives substituted by carboxyl, methylcarboxyl or benzoxymethyl with lithium aluminum hydride, or a hydrolysis to 2-hydroxymethyl-TEDA. 2-Hydroxymethyl-TEDA can subsequently be reacted with acetic anhydride to give the corresponding acetate.

DE-A 30 48 031 relates to a process for preparing substituted pyrazines. In this process, dihydropyrazines are reacted with carbonyl derivatives in the presence of a base to give substituted pyrazine. Suitable carbonyl compounds also include carbonyl compounds which comprise a double bond, such as terpene aldehydes, unsaturated aliphatic aldehydes, unsaturated aliphatic ketones or furan aldehydes. Especially owing to the use of strong organic bases such as alkali metal alkoxides (alcoholate), alkali metal hydrides or alkali metal amides, the process described in DE-A 30 48 031 does not afford TEDA derivatives. The reason for this different reactivity should be sought in a different reaction mechanism which underlies the reaction described in DE-A 30 48 031.

The reaction described in DE-A 30 48 031 is based on the following mechanism: First, the strong base used, for example an alkoxide, abstracts a proton in the saturated 5 position of the 5,6-dihydropyrazine to form the corresponding acid, for example of the appropriate alcohol. The dihydropyrazine anion formed then nucleophilically attacks the carbonyl compound and thus forms a 5-[1′-hydroxy-1′-(substituent)methyl]-5,6-dihydropyrazine intermediate. This can eliminate water and forms a 5-[1′-(substituent)-methylidene]-5,6-dihydropyrazine intermediate 1′-substituted on the exocyclic carbon atom. This in turn rearranges with aromatization via a 1,3-H shift rearrangement to the end product, a 5-[1′-(substituent)methyl]pyrazine.

The fact that the reaction proceeds via a deprotonation of the dihydropyrazine and not the other way round, i.e. via formation of an enolate from the carbonyl compound, is demonstrated by the fact that it is also possible to use aldehydes which do not have a proton on the carbon atom adjacent to the carbonyl group, for example benzaldehyde, and consequently cannot form an enolate. The reaction described in DE-A 30 48 031 of dihydropyrazines with carbonyl compounds, and if appropriate also unsaturated carbonyl compounds, accordingly leads to products in which the atoms of the former carbonyl compound cannot be joined to the nitrogen atoms of the dihydropyrazine.

It is therefore an object of the invention to provide a novel, simplified process for preparing TEDA derivatives. In the context of the present invention, unsubstituted TEDA is also comprised by the term “TEDA derivative”.

According to the invention, this object is achieved by a process for preparing triethylenediamine (TEDA) derivatives, comprising the following steps:
a) reacting a dihydropyrazine with an olefin,
b) if appropriate hydrogenating after step a).

In contrast to the process described in DE-A 30 48 031, there is an additional ring closure (“bridge formation”) in the process according to the invention, which joins the two carbon atoms of the olefin double bond to the two ring nitrogen atoms of the dihydropyrazine through the reaction.

The process according to the invention advantageously allows TEDA and TEDA derivatives to be prepared, especially those TEDA derivatives which comprise substituents with functional groups. Substituents with functional groups shall be understood to mean those substituents which have at least one heteroatom such as halogen, S, P, O or N. TEDA derivatives with substituents which comprise functional groups are suitable in a particularly advantageous manner as a catalyst for polyurethane preparation (PU catalyst). The polyurethanes prepared in this way, especially polyurethane foams, are notable in that the catalysts used do not outgas from the polyurethane, since they are chemically incorporated into the corresponding polyurethane, especially into polyurethane foam, or because they possess an elevated vapor pressure.

It is additionally advantageous that, to obtain such a polyurethane or polyurethane foam, greater amounts or significantly greater amounts of PU catalyst than are customary in systems already established to date, especially unsubstituted TEDA, are not required.

The process according to the invention for preparing TEDA derivatives will be characterized in detail hereinafter.

In step a), a dihydropyrazine is reacted with an olefin, which in each case joins a carbon atom of the olefin double bond to a ring nitrogen atom of the dihydropyrazine in each case through the reaction. With regard to the selection of dihydropyrazine and of olefin, there are in principle no restrictions; both reactants may either be substituted or unsubstituted. These reactants are commercially available or can be prepared by methods known to those skilled in the art. Details of the synthesis of the dihydropyrazine reactants are described in detail hereinafter.

### Synthesis of the Dihydropyrazines

Processes for preparing dihydropyrazines are known to those skilled in the art. A good overview of the preparation of substituted dihydropyrazines is given in Flament, I., Stoll, M., Helvetica Chimica Acta 1967, Vol. 50 (7), No. 180, pages 1754-1758. In addition, the synthesis of dihydropyrazines is also described in DE 103 21 565 A1 (albeit with the aim of subsequently dehydrogenating them to the corresponding pyrazines).

The reactant synthesis proceeding from (optionally substituted) ethylenediamine(s) and dicarbonyl compounds to give the dihydropyrazines can be carried out in various solvents. Theoretically, all organic solvents and water are suitable. It is less preferred to use protic solvents, for example 1,2-propanediol or water, because the reaction in these solvents leads to poorer dihydropyrazine selectivities and to an enhanced tendency to form polymers. Moreover, the use of diethyl ether is chemically viable but inadvisable on the industrial scale for safety reasons (low boiling point, autoxidizability).

Optimal conversion and selectivity have been achieved by performing the reaction in a moderately polar but water-immiscible solvent. In addition, N₂ should be employed as a protective gas.

Particular preference is given to tert-butyl methyl ether (MTBE) as an optimal solvent. This solvent possesses similar chemical properties to diethyl ether (dissolution capacity, polarity and mixing behavior with organic solvents or water), but a higher boiling point and no autoxidizability, and can thus be handled more safely. Moreover, advantages can be obtained in MTBE through the low miscibility with water, because the water formed in the reaction separates out as a second phase and thus no longer disrupts the reaction, and because almost all by-products and residues of ethylene-diamine are additionally present in the aqueous phase and can be removed very easily after the end of the reaction.

Ethylenediamine or an EDA derivative is initially charged in a portion of MTBE, and the dicarbonyl compound (in an equimolar amount) is slowly added dropwise in MTBE. This forms an insoluble intermediate which decomposes to product, EDA and water in the course of subsequent heating to ambient temperature or higher. The intermediate complicates the homogeneous stirring to a high degree; a comparatively large amount of solvent is therefore used. In the case of too small an amount of solvent, the mixture solidifies; lumps which still comprise dicarbonyl compound form. These lumps are heated rapidly and deliquesce to form a viscous black mass. It is therefore not possible to work without solvent.

Excessively high temperature and the water which forms promote the polymerization of the product. tert-Butyl methyl ether (MTBE) was found to be ideal, since the water separates out as a second phase and can be removed together with the by-products. The colorless ethereal phase is dried and concentrated under reduced pressure, and the product mixture is subsequently fractionally distilled under reduced pressure. The product is air-stable at −20° C., but polymerizes at ambient temperature in the presence of oxygen.

A particularly positive aspect of this reaction regime is the fact that it is possible to work with equimolar amounts of EDA (or EDA derivative) to dicarbonyl compound because an excess of EDA (derivative) in the subsequent stage can have a disruptive effect. For example, an EDA (derivative), when the subsequent stage is the reaction with an acrylic acid derivative, can likewise carry out a 1,4 addition on the olefin and thus form undesired by-products. Particular preference is therefore given to working with equimolar amounts of EDA derivative to dicarbonyl compound. It is also possible to use one reactant in an excess of from 1:20 to 20:1, but this method is advantageous only when the reactant present in excess can subsequently be removed, for example by distillation, precipitation or other methods known to those skilled in the art. Since, however, dihydropyrazines can be polymerized under thermal stress, it is particularly advantageous when the removal can be dispensed with owing to the use of equimolar amounts.

The removal of any residue of EDA or EDA derivative via a second aqueous phase which forms is particularly advantageous. This removability arises from the particularly advantageous use of a moderately polar, aprotic, water-immiscible solvent, for example MTBE. Particular preference is therefore given to performing the synthesis of the dihydropyrazines in MTBE.

The synthesis of the dihydropyrazines can be carried out between −80° C. and 80° C. Preference is given to performing the reaction between −20° C. and 60° C., particular preference to performing it between 0° C. and 50° C.

The reaction pressure may be between 0.5 and 250 bar (absolute). Preference is given to working at standard pressure.

Particular preference is given to working without an additional added catalyst in the synthesis of the dihydropyrazines because the reactivity of the dicarbonyl compounds and of the ethylenediamine derivatives is itself very high.

Step a: Reaction of the Dihydropyrazines with the Olefin

In step a), TEDA derivatives which have a carbon-carbon double bond in the TEDA base skeleton (unsaturated TEDA derivatives) are prepared. In order to obtain TEDA derivatives which have a fully saturated TEDA base skeleton, it is possible if appropriate to carry out a step b) in which the product obtained in step a) is subjected to a hydrogenation.

The reaction of the dihydropyrazine with the olefin can be carried out in various solvents. Theoretically, all organic solvents and water are suitable. Examples of suitable solvents are methanol, 1,2-propanediol, dioxane, tetrahydrofuran or MTBE.

It is advantageous to use the same solvent which is used in the synthesis of the dihydropyrazines (e.g. MTBE), because no solvent exchange is then required after the synthesis of the dihydropyrazines. It is particularly advantageous to use relatively high-boiling, aprotic solvents, because higher reaction temperatures, shorter reaction times and hence a more economic space-time yield are also possible therein. The best results in this regard are obtained with dioxane as the solvent.

A catalyst can be used. Preference is given to performing step a) in the absence of bases. Particular preference is given to working without addition of a catalyst, since the reaction can also be controlled thermally.

The reaction can be carried out at temperatures of −50° C. to 200° C. Preference is given to performing the reaction at a reaction temperature between 0° C. and 150° C. The reaction temperature is more preferably from 60° C. to 130° C.

The reaction pressure may be between 0.5 and 250 bar (absolute). Preference is given to working at standard pressure.

The molar ratio of dihydropyrazine to olefin can be varied from 20:1 to 1:20. It is particularly advantageous to use equimolar amounts.

The reaction can be explained by the mechanism of a cycloaddition between a diene and a dienophile (Diels-Alder reaction) or as a Michael addition. The choice of substituents is therefore substantially as desired, since the diene skeleton of the dihydropyrazine and the olefinic double bond in particular are involved in the reaction. However, the reaction rate and chemoselectivity of cycloadditions is often dependent on the electronic conditions in the two reactants.

As an example which does not restrict the scope of the invention, the reaction of 2,3-dimethyl-5,6-dihydropyrazine with ethyl acrylate is given. This proceeds particularly advantageously because the acrylate, as an electron-deficient dienophile, is a good match for the diene used. The same applies, for example, to maleic esters. For the same dihydropyrazine, in contrast, electron-richer dienophiles (e.g. methyl vinyl ether) are less suitable as reactants and therefore react less efficiently, more slowly or less selectivity with one another. Conversely, as is known to the person skilled in the art, electron-rich dienophiles react readily with the dihydropyrazine when the electronic conditions are matched through appropriate substituents.

If required, the properties of the substituents on the two reactants can be varied by introducing protecting groups, derivatizing and umpolung, in order to achieve the desired reactivity. The introduction of protecting groups is known to those skilled in the art.

Step b: Hydrogenation of the Reaction Product from a)

The hydrogenation in step b) is effected by methods known to those skilled in the art. For example, the hydrogenation can be carried out directly after step a) without isolating the unsaturated TEDA derivative obtained in step a). The products obtained in steps a) and b) can be purified and isolated by methods known to those skilled in the art.

When protected reactants have been used in step a), the corresponding protecting groups can be removed again by methods known to those skilled in the art after step a) or, if appropriate, after step b) (deprotection step c)). If appropriate, this deprotection can also be carried out together with the hydrogenation in step b).

In the context of the present invention—unless stated otherwise—the following also applies to all reactants used and to all products obtained:

Alkyl radicals (C₁-C₁₀-alkyl; this abbreviation means that the corresponding alkyl radical has from 1 to 10 carbon atoms) may be either linear or branched, acyclic or cyclic, and saturated or unsaturated. This is also true when they are part of another group, for example alkoxy groups (C₁-C₁₀-alkyl-O—), alkoxycarbonyl groups or aminoalkyl groups, or when they are substituted. Alkyl accordingly also comprises alkylene radicals (—(CH₂)ₙ— where n is, for example, from 1 to 10). For example, alkylamino-(C₁-C₁₀-alkoxy) means that a C₁-C₁₀-alkoxy radical is in turn substituted by an alkylamino radical.

Examples of alkyl groups are: methyl, ethyl, propyl, butyl, pentyl, hexyl, heptyl, octyl, nonyl or decyl. This also comprises both the n-isomers of these radicals and branched isomers, for example isopropyl, isobutyl, isopentyl, sec-butyl, tert-butyl, neopentyl, 3,3-dimethylbutyl, 2-ethylhexyl, etc. Unless stated otherwise, the term “alkyl” additionally also includes alkyl radicals which are unsubstituted or optionally substituted by one or more further radicals, for example from 1 to 10 identical or different radicals, for example hydroxyl, amino, alkylamino, dialkylamino, aryl, heteroaryl, alkoxy or halogen. The additional substituents may occur in any desired position in the alkyl radical. In addition, the term “alkyl” also comprises cycloalkyl and cycloalkylalkyl (alkyl which is in turn substituted by cycloalkyl), where cycloalkyl has at least 3 carbon atoms. Examples of cycloalkyl radicals are: cyclopropyl, cyclobutyl, cyclopentyl, cyclohexyl, cycloheptyl, cyclooctyl, cyclononyl and cyclodecyl. If appropriate, they may also be polycyclic ring systems, such as decalinyl, norbornanyl, bornanyl or adamantanyl. The cycloalkyl radicals may be unsubstituted or optionally substituted by one or more further radicals, as listed above by way of example for the alkyl radicals.

Halogen is fluorine, chlorine, bromine or iodine.

Aryl is a 5- to 14-membered, aromatic mono-, bi- or tricycle. The aryl radical is thus derived from mono-, bi- or tricyclic aromatics which do not comprise any ring heteroatoms. When they are not monocyclic systems, the second or third ring may also be present in the saturated or in the partly unsaturated form, provided that the particular forms are known and stable. If appropriate, aryl may also in turn be at least monosubstituted, for example by halogen, alkyl or alkoxy. Examples of aryl are: phenyl, naphthyl, indanyl, 1,2-dihydronaphthenyl or 1,2,3,4-tetrahydronaphthyl. Aryl is preferably phenyl.

A preferred embodiment of the present invention relates to a process for preparing triethylenediamine (TEDA) derivatives of the general formulae (Ia) and (Ib)

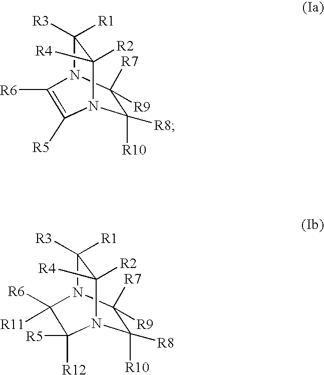

comprising the following steps:
a) reacting a dihydropyrazine (II) with an olefin (III) to obtain the TEDA derivative (Ia)

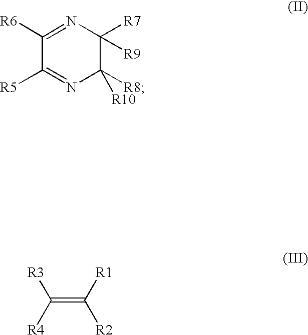

b) if appropriate hydrogenating the TEDA derivative (Ia) to obtain the TEDA derivative (Ib),
   where, in the formulae (I) to (III):

All carbon atoms may have R or S configuration and E or Z configuration. The representation shown in the structural formulae does not imply a representation of the absolute configuration (for example endo or exo position or an upward/downward orientation of a particular substituent) on one carbon atom or the absolute configuration of a double bond; the drawing should only be understood as an illustration of connectivity, i.e. which substituents may be situated on which carbon atom.

R1 to R10 are each independently selected from

H, halogen, —(C₁-C₁₀-alkyl)-(R14)ᵤ, —C(O)—R13, —CN, —O-aryl(R14)ᵤ, —(C₁-C₁₀-alkyl)-O—C(O)R14, —O—[(C₁-C₁₀-alkyl)-(R14)ᵤ]ₚH_{q}, —N—[(C₁-C₁₀-alkyl)-(R14)ᵤ]ₚH_{g}Aᵣ, —P—[(C₁-C₁₀-alkyl)-(R14)ᵤ]ₚH_{q}Aᵣ or —S—[(C₁-C₁₀-alkyl)-(R14)ᵤ]ₚH_{q}; u is from 0 to 10. When u=0, this means that the appropriate (C₁-C₁₀-alkyl) fragment of the substituents R1 to R10 is unsubstituted. When u≧1, the appropriate (C₁-C₁₀-alkyl) fragment is substituted by u R14 substituents, where, in the case that u>1, the particular R14 radicals are each independently substituted. The R14 radicals may be in any desired position in the (C₁-C₁₀-alkyl) fragment, the upper limit of R14 radicals being fixed by the number of hydrogen atoms in the appropriate (C₁-C₁₀-alkyl) fragment.

If a product of the formula (Ib) has originated from the hydrogenation of a product of the formula (Ia), the substituents R11 and R12 are typically each H. If appropriate, the substituents R11 and R12 can be modified after the hydrogenation by methods known to those skilled in the art according to the definitions of R1 to R10.

R13 is H, hydroxyl, amino, aryl, C₁-C₁₀-alkoxy, amino(C₁-C₁₀-alkoxy), alkylamino-(C₁-C₁₀-alkoxy), dialkylamino(C₁-C₁₀-alkoxy), amino(C₁-C₁₀-alkyl), aminoaryl, —NH(C₁-C₁₀-alkyl), —N(C₁-C₁₀-alkyl)₂, —NH-aryl, —N-(aryl)₂, halogen, hydroxyaryl, —O-aryl or —O—(C₁-C₁₀-alkyl)aryl;
R14 is hydroxyl, amino, aryl, C₁-C₁₀-alkoxy, amino(C₁-C₁₀-alkoxy), alkylamino(C₁-C₁₀-alkoxy), dialkylamino(C₁-C₁₀-alkoxy), amino(C₁-C₁₀-alkyl), —NH(C₁-C₁₀-alkyl), —N(C₁-C₁₀-alkyl)₂, hydroxyaryl, aminoaryl, —NH-aryl, —N(aryl)₂ᵢ halogen, —PH-aryl, —P(aryl)₂, —O-aryl or —O—(C₁-C₁₀-alkyl)aryl;

If the [(C₁-C₁₀-alkyl)-(R14)ᵤ] radical is bonded to nitrogen or phosphorus:

p is from 0 to 3, where q may likewise be from 0 to 3. For the sum of p+q:

If p is 0, q is 2 or 3.

If p is 1, q is 1 or 2.

If p is 2, q is 0 or 1.

If the sum of p and q is 2, the heteroatom is uncharged, and therefore no counteranion A is present and thus r=0.

If the sum of p and q is 3, the heteroatom is provided with a positive charge; therefore, a counteranion A is present and thus r=1. The corresponding TEDA derivative is then present as a salt. Preferably, however, the TEDA derivatives are present as an uncharged molecule, i.e. r is 0.

A may be any anion; A is preferably selected from halogen, sulfate, sulfite, nitrate, nitrite, phosphate, phosphite, hypophosphite, formate, acetate, propionate, oxalate and citrate. Halogen is especially chlorine.

If the [(C₁-C₁₀-alkyl)-(R14)ᵤ] radical is bonded to oxygen or sulfur:

p is 0 or 1.

If p is 0, q is 1.

If p is 1, q is 0.

Preferably, at least one of the substituents R1 to R10 in the dihydropyrazine (I) or in the olefin (III) is selected from

—[(C₁-C₁₀-alkyl)-(R14)ᵤ], —O—[(C₁-C₁₀-alkyl)-(R14)ᵤ], —N[(C₁-C₁₀-alkyl)-(R14)ᵤ]₂, —NH[(C₁-C₁₀-alkyl)-(R14)ᵤ]—C(O)—R13 and —CN,
where R13 and R14 are each independently hydroxyl, C₁-C₁₀-alkoxy, —NH₂, —NH(C₁-C₁₀-alkyl), —N(C₁-C₁₀-alkyl)₂, —O-aryl or —O—(C₁-C₁₀-alkyl)aryl, and u is from 0 to 10.

More preferably, the dihydropyrazine (II) is selected from 2,3-dihydropyrazine, 2-methyl-5,6-dihydropyrazine, 2-ethyl-5,6-dihydropyrazine, 2-propyl-5,6-dihydropyrazine, 2,3-dimethyl-5,6-dihydropyrazine, 2,3-diethyl-5,6-dihydropyrazine, 2-ethyl-3-methyl-5,6-dihydropyrazine, 2,5-dimethyl-5,6-dihydropyrazine, 2,6-dimethyl-5,6-dihydropyrazine, 2,3,5-trimethyl-5,6-dihydropyrazine, 2-hydroxy-5,6-dihydropyrazine, 2-methyl-3-hydroxy-5,6-dihydropyrazine, 2-methyl-5-hydroxy-5,6-dihydropyrazine, 2-methyl-6-hydroxy-5,6-dihydropyrazine, 2-dimethylamino-5,6-dihydropyrazine, 5-dimethylamino-5,6-dihydropyrazine or 2-methyl-3-dimethylamino-5,6-dihydropyrazine.

More preferably, the olefin (III) is selected from ethylene, propylene, butylene, hydroxypropylene, hydroxybutylene, vinyl methyl ketone, acrylic acid, methyl acrylate, ethyl acrylate, propyl acrylate, vinyl methyl ether, dimethylaminoethyl vinyl ether, 2-hydroxyethyl vinyl ether, 1-hydroxyethyl vinyl ether, allyl methyl ketone, 3-hydroxybut-1-ene, 3-hydroxypent-1-ene, 3-hydroxyhex-1-ene, 4-hydroxybut-1-ene, 4-hydroxypent-1-ene, 4-hydroxyhex-1-ene, 5-hydroxypent-1-ene, 5-hydroxyhex-1-ene, 6-hydroxyhex-1-ene, 4-hydroxypent-2-ene, 4-hydroxyhex-2-ene, 2-hydroxybut-3-ene, 4-hydroxypent-2-ene, 4-hydroxyhex-2-ene, 5-hydroxypent-2-ene, 5-hydroxyhex-2-ene, 6-hydroxyhex-2-ene, allyl acetate, maleic acid, dimethyl maleate, diethyl maleate, monomethyl monoethyl maleate, maleic anhydride, fumaric acid, dimethyl fumarate, diethyl fumarate, monomethyl monoethyl fumarate or fumaric anhydride.

The TEDA derivative (Ia) is preferably a compound of the formula (Ia1)

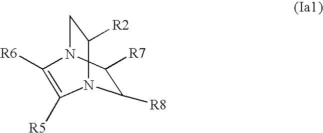

where R2, R7 and R8 are each independently H, —OH, —(C₁-C₃-alkyl)-OH, —(C₁-C₃-alkyl)-O—(C₁-C₃-alkyl), —CN, —O-phenyl, —(C₁-C₃-alkyl)-O-phenyl, C₁-C₃-alkoxy, —C(O)(C₁-C₃-alkoxy), —C(O)OH, —N(CH₃)₂, —NH(CH₃), —NH₂, —(C₁-C₃-alkyl)-N(CH₃)₂, —(C₁-C₃-alkyl)-NH(CH₃), —(C₁-C₃-alkyl)-NH₂, or
—(C₁-C₃-alkyl)-O—C(O)(C₁-C₃-alkoxy),
R5 and R6 are each independently H, C₁-C₃-alkyl, —C(O)OH, —C(O)(C₁-C₃-alkoxy) or —(C₁-C₃-alkyl)-O—(C₁-C₃-alkyl),
and at least one of the substituents R2, R7 and R8 is not H.

In one embodiment, particularly preferred TEDA derivatives (Ia) are selected from

2-hydroxy-1,4-diazabicyclo[2.2.2]oct-5-ene, 2-carboxy-1,4-diazabicyclo[2.2.2]oct-5-ene, 2-hydroxycarbonyl-1,4-diazabicyclo[2.2.2]oct-5-ene, 2-formyloxy-1,4-diazabicyclo-[2.2.2]oct-5-ene, 2-acetoxy-1,4-diazabicyclo[2.2.2]oct-5-ene, 2-propionyloxy-1,4-diazabicyclo[2.2.2]oct-5-ene, 2-hydroxy-5-methyl-1,4-diazabicyclo[2.2.2]oct-5-ene, 2-carboxy-5-methyl-1,4-diazabicyclo[2.2.2]oct-5-ene, 2-hydroxycarbonyl-5-methyl-1,4-diazabicyclo[2.2.2]oct-5-ene, 2-formyloxy-5-methyl-1,4-diazabicyclo[2.2.2]oct-5-ene, 2-acetoxy-5-methyl-1,4-diazabicyclo[2.2.2]oct-5-ene, 2-propionyloxy-5-methyl-1,4-diazabicyclo[2.2.2]oct-5-ene, 2-hydroxy-6-methyl-1,4-diazabicyclo[2.2.2]oct-5-ene, 2-carboxy-6-methyl-1,4-diazabicyclo[2.2.2]oct-5-ene, 2-hydroxycarbonyl-6-methyl-1,4-diazabicyclo[2.2.2]oct-5-ene, 2-formyloxy-6-methyl-1,4-diazabicyclo[2.2.2]oct-5-ene, 2-acetoxy-6-methyl-1,4-diazabicyclo[2.2.2]oct-5-ene, 2-propionyloxy-6-methyl-1,4-diazabicyclo[2.2.2]oct-5-ene, 2-hydroxy-5,6-dimethyl-1,4-diazabicyclo[2.2.2]oct-5-ene, 2-carboxy-5,6-dimethyl-1,4-diazabicyclo[2.2.2]oct-5-ene, 2-hydroxycarbonyl-5,6-dimethyl-1,4-diazabicyclo[2.2.2]oct-5-ene, 2-formyloxy-5,6-dimethyl-1,4-diazabicyclo[2.2.2]oct-5-ene, 2-acetoxy-5,6-dimethyl-1,4-diazabicyclo[2.2.2]oct-5-ene, 2-propionyloxy-5,6-dimethyl-1,4-diazabicyclo[2.2.2]oct-5-ene, 2-hydroxymethyl-1,4-diazabicyclo[2.2.2]oct-5-ene, 2-hydroxymethyl-5-methyl-1,4-diazabicyclo[2.2.2]oct-5-ene, 2-hydroxymethyl-6-methyl-1,4-diazabicyclo[2.2.2]oct-5-ene, 2-hydroxymethyl-5,6-dimethyl-1,4-diazabicyclo[2.2.2]oct-5-ene, 2-(1′-hydroxyethyl)-1,4-diazabicyclo-[2.2.2]oct-5-ene, 2-(1′-hydroxyethyl)-5-methyl-1,4-diazabicyclo[2.2.2]oct-5-ene, 2-(1′-hydroxyethyl)-6-methyl-1,4-diazabicyclo[2.2.2]oct-5-ene, 2-(1′-hydroxyethyl)-5,6-dimethyl-1,4-diazabicyclo[2.2.2]oct-5-ene, 2-(2′-hydroxyethyl)-1,4-diazabicyclo-[2.2.2]oct-5-ene, 2-(2′-hydroxyethyl)-5-methyl-1,4-diazabicyclo[2.2.2]oct-5-ene, 2-(2′-hydroxyethyl)-6-methyl-1,4-diazabicyclo[2.2.2]oct-5-ene, 2-(2′-hydroxyethyl)-5,6-dimethyl-1,4-diazabicyclo[2.2.2]oct-5-ene, 2-methoxycarbonyl-1,4-diazabicyclo-[2.2.2]oct-5-ene, 2-methoxycarbonyl-5-methyl-1,4-diazabicyclo[2.2.2]oct-5-ene, 2-methoxycarbonyl-6-methyl-1,4-diazabicyclo[2.2.2]oct-5-ene, 2-methoxycarbonyl-5,6-dimethyl-1,4-diazabicyclo[2.2.2]oct-5-ene, 2-methoxycarbonyl-5-methyl-6-ethyl-1,4-diazabicyclo[2.2.2]oct-5-ene, 2-methoxycarbonyl-5-ethyl-6-methyl-1,4-diazabicyclo-[2.2.2]oct-5-ene, 2-ethoxycarbonyl-1,4-diazabicyclo[2.2.2]oct-5-ene, 2-ethoxycarbonyl-5-methyl-1,4-diazabicyclo[2.2.2]oct-5-ene, 2-ethoxycarbonyl-6-methyl-1,4-diazabicyclo[2.2.2]oct-5-ene, 2-ethoxycarbonyl-5,6-dimethyl-1,4-diazabicyclo[2.2.2]oct-5-ene, 2-ethoxycarbonyl-5-methyl-6-ethyl-1,4-diazabicyclo[2.2.2]oct-5-ene, 2-ethoxycarbonyl-5-ethyl-6-methyl-1,4-diazabicyclo[2.2.2]oct-5-ene, 2-acetoxymethyl-1,4-diazabicyclo[2.2.2]oct-5-ene, 2-acetoxymethyl-5-methyl-1,4-diazabicyclo[2.2.2]oct-5-ene, 2-acetoxymethyl-6-methyl-1,4-diazabicyclo[2.2.2]oct-5-ene, 2-acetoxymethyl-5,6-dimethyl-1,4-diazabicyclo[2.2.2]oct-5-ene, 2-(dimethylamino)-1,4-diazabicyclo-[2.2.2]oct-5-ene, 2-(dimethylamino)-5-methyl-1,4-diazabicyclo[2.2.2]oct-5-ene, 2-(dimethylamino)-6-methyl-1,4-diazabicyclo[2.2.2]oct-5-ene, 2-(dimethylamino)-5,6-dimethyl-1,4-diazabicyclo[2.2.2]oct-5-ene, 2-(dimethylaminomethyl)-1,4-diazabicyclo[2.2.2]oct-5-ene, 2-(dimethylaminomethyl)-5-methyl-1,4-diazabicyclo[2.2.2]oct-5-ene, 2-(dimethylaminomethyl)-6-methyl-1,4-diazabicyclo[2.2.2]oct-5-ene, 2-(dimethylaminomethyl)-5,6-dimethyl-1,4-diazabicyclo[2.2.2]oct-5-ene, 2-(dimethylaminoethoxy)-1,4-diazabicyclo[2.2.2]oct-5-ene, 2-(dimethylaminoethoxy)-5-methyl-1,4-diazabicyclo-[2.2.2]oct-5-ene, 2-(dimethylaminoethoxy)-6-methyl-1,4-diazabicyclo[2.2.2]oct-5-ene, 2-(dimethylaminoethoxy)-5,6-dimethyl-1,4-diazabicyclo[2.2.2]oct-5-ene, 2-[(dimethylaminoethoxy)carbonyl]-1,4-diazabicyclo[2.2.2]oct-5-ene, 2-[(dimethylaminoethoxy)-carbonyl]-5-methyl-1,4-diazabicyclo[2.2.2]oct-5-ene, 2-[(dimethylaminoethoxy)-carbonyl]-6-methyl-1,4-diazabicyclo[2.2.2]oct-5-ene, 2-[(dimethylaminoethoxy)-carbonyl]-5,6-dimethyl-1,4-diazabicyclo[2.2.2]oct-5-ene, 2-(1′-hydroxypropyl)-1,4-diazabicyclo[2.2.2]oct-5-ene, 2-(1′-hydroxypropyl)-5-methyl-1,4-diazabicyclo[2.2.2]oct-5-ene, 2-(1′-hydroxypropyl)-6-methyl-1,4-diazabicyclo[2.2.2]oct-5-ene, 2-(1′-hydroxypropyl)-5,6-dimethyl-1,4-diazabicyclo[2.2.2]oct-5-ene, 2-(2′-hydroxypropyl)-1,4-diazabicyclo-[2.2.2]oct-5-ene, 2-(2′-hydroxypropyl)-5-methyl-1,4-diazabicyclo[2.2.2]oct-5-ene, 2-(2′-hydroxypropyl)-6-methyl-1,4-diazabicyclo[2.2.2]oct-5-ene, 2-(2′-hydroxypropyl)-5,6-dimethyl-1,4-diazabicyclo[2.2.2]oct-5-ene, 2-(3′-hydroxypropyl)-1,4-diazabicyclo[2.2.2]oct-5-ene, 2-(3′-hydroxypropyl)-5-methyl-1,4-diazabicyclo[2.2.2]oct-5-ene, 2-(3′-hydroxypropyl)-6-methyl-1,4-diazabicyclo[2.2.2]oct-5-ene, 2-(3′-hydroxypropyl)-5,6-dimethyl-1,4-diazabicyclo[2.2.2]oct-5-ene, 2,3-bis(hydroxymethyl)-1,4-diazabicyclo-[2.2.2]oct-5-ene, 2,3-bis(hydroxymethyl)-5-methyl-1,4-diazabicyclo[2.2.2]oct-5-ene, 2,3-bis(hydroxymethyl)-6-methyl-1,4-diazabicyclo[2.2.2]oct-5-ene, 2,3-bis(hydroxymethyl)-5,6-dimethyl-1,4-diazabicyclo[2.2.2]oct-5-ene, 2,3-bis(ethoxycarbonyl)-1,4-diazabicyclo[2.2.2]oct-5-ene, 2,3-bis(ethoxycarbonyl)-5-methyl-1,4-diazabicyclo-[2.2.2]oct-5-ene, 2,3-bis(ethoxycarbonyl)-6-methyl-1,4-diazabicyclo[2.2.2]oct-5-ene and 2,3-bis(ethoxycarbonyl)-5,6-dimethyl-1,4-diazabicyclo[2.2.2]oct-5-ene.

The TEDA derivative (Ib) is preferably a compound of the formula (Ib1),

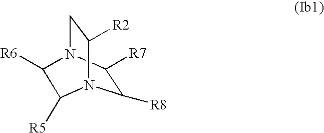

where R2, R7 and R8 are each independently H, —OH, —(C₁-C₃-alkyl)-OH, —(C₁-C₃-alkyl)-O—(C₁-C₃-alkyl), —CN, —O-phenyl, —(C₁-C₃-alkyl)-O-phenyl, C₁-C₃-alkoxy, —C(O)(C₁-C₃-alkoxy), —C(O)OH, —N(CH₃)₂, —NH(CH₃), —NH₂, —(C₁-C₃-alkyl)-N(CH₃)₂, —(C₁-C₃-alkyl)-NH(CH₃), —(C₁-C₃-alkyl)-NH₂, or
—(C₁-C₃-alkyl)-O—C(O)(C₁-C₃-alkoxy),
R5 and R6 are each independently H, —C(O)OH, —C(O)(C₁-C₃-alkoxy) or —(C₁-C₃-alkyl)-O—(C₁-C₃-alkyl),
and at least one of the substituents R2, R7 and R8 is not H.

In one embodiment, particularly preferred TEDA derivatives (Ib) are selected from

2-hydroxy-1,4-diazabicyclo[2.2.2]octane, 2-carboxy-1,4-diazabicyclo[2.2.2]octane, 2-hydroxycarbonyl-1,4-diazabicyclo[2.2.2]octane, 2-formyloxy-1,4-diazabicyclo[2.2.2]octane, 2-acetoxy-1,4-diazabicyclo[2.2.2]octane, 2-propionyloxy-1,4-diazabicyclo[2.2.2]octane, 2-hydroxy-5-methyl-1,4-diazabicyclo[2.2.2]octane, 2-carboxy-5-methyl-1,4-diazabicyclo[2.2.2]octane, 2-hydroxycarbonyl-5-methyl-1,4-diazabicyclo[2.2.2]octane, 2-formyloxy-5-methyl-1,4-diazabicyclo[2.2.2]octane, 2-acetoxy-5-methyl-1,4-diazabicyclo[2.2.2]octane, 2-propionyloxy-5-methyl-1,4-diazabicyclo[2.2.2]octane, 2-hydroxy-6-methyl-1,4-diazabicyclo[2.2.2]octane, 2-carboxy-6-methyl-1,4-diazabicyclo[2.2.2]octane, 2-hydroxycarbonyl-6-methyl-1,4-diazabicyclo-[2.2.2]octane, 2-formyloxy-6-methyl-1,4-diazabicyclo[2.2.2]octane, 2-acetoxy-6-methyl-1,4-diazabicyclo[2.2.2]octane, 2-propionyloxy-6-methyl-1,4-diazabicyclo[2.2.2]octane, 2-hydroxy-5,6-dimethyl-1,4-diazabicyclo[2.2.2]octane, 2-carboxy-5,6-dimethyl-1,4-diazabicyclo[2.2.2]octane, 2-hydroxycarbonyl-5,6-dimethyl-1,4-diazabicyclo-[2.2.2]octane, 2-formyloxy-5,6-dimethyl-1,4-diazabicyclo[2.2.2]octane, 2-acetoxy-5,6-dimethyl-1,4-diazabicyclo[2.2.2]octane, 2-propionyloxy-5,6-dimethyl-1,4-diazabicyclo[2.2.2]octane, 2-hydroxymethyl-1,4-diazabicyclo[2.2.2]octane, 2-hydroxymethyl-5-methyl-1,4-diazabicyclo[2.2.2]octane, 2-hydroxymethyl-6-methyl-1,4-diazabicyclo-[2.2.2]octane, 2-hydroxymethyl-5,6-dimethyl-1,4-diazabicyclo[2.2.2]octane, 2-(1′-hydroxyethyl)-1,4-diazabicyclo[2.2.2]octane, 2-(1′-hydroxyethyl)-5-methyl-1,4-diazabicyclo[2.2.2]octane, 2-(1′-hydroxyethyl)-6-methyl-1,4-diazabicyclo-[2.2.2]octane, 2-(1′-hydroxyethyl)-5,6-dimethyl-1,4-diazabicyclo[2.2.2]octane, 2-(2′-hydroxyethyl)-1,4-diazabicyclo[2.2.2]octane, 2-(2′-hydroxyethyl)-5-methyl-1,4-diazabicyclo[2.2.2]octane, 2-(2′-hydroxyethyl)-6-methyl-1,4-diazabicyclo-[2.2.2]octane, 2-(2′-hydroxyethyl)-5,6-dimethyl-1,4-diazabicyclo[2.2.2]octane, 2-methoxycarbonyl-1,4-diazabicyclo[2.2.2]octane, 2-methoxycarbonyl-5-methyl-1,4-diazabicyclo[2.2.2]octane, 2-methoxycarbonyl-6-methyl-1,4-diazabicyclo[2.2.2]-octane, 2-methoxycarbonyl-5,6-dimethyl-1,4-diazabicyclo[2.2.2]octane, 2-methoxycarbonyl-5-methyl-6-ethyl-1,4-diazabicyclo[2.2.2]octane, 2-methoxycarbonyl-5-ethyl-6-methyl-1,4-diazabicyclo[2.2.2]octane, 2-ethoxycarbonyl-1,4-diazabicyclo[2.2.2]-octane, 2-ethoxycarbonyl-5-methyl-1,4-diazabicyclo[2.2.2]octane, 2-ethoxycarbonyl-6-methyl-1,4-diazabicyclo[2.2.2]octane, 2-ethoxycarbonyl-5,6-dimethyl-1,4-diazabicyclo[2.2.2]octane, 2-ethoxycarbonyl-5-methyl-6-ethyl-1,4-diazabicyclo[2.2.2]octane, 2-ethoxycarbonyl-5-ethyl-6-methyl-1,4-diazabicyclo[2.2.2]octane, 2-acetoxymethyl-1,4-diazabicyclo[2.2.2]octane, 2-acetoxymethyl-5-methyl-1,4-diazabicyclo[2.2.2]octane, 2-acetoxymethyl-6-methyl-1,4-diazabicyclo[2.2.2]octane, 2-acetoxymethyl-5,6-dimethyl-1,4-diazabicyclo[2.2.2]octane, 2-(dimethylamino)-1,4-diazabicyclo[2.2.2]-octane, 2-(dimethylamino)-5-methyl-1,4-diazabicyclo[2.2.2]octane, 2-(dimethylamino)-6-methyl-1,4-diazabicyclo[2.2.2]octane, 2-(dimethylamino)-5,6-dimethyl-1,4-diazabicyclo[2.2.2]octane, 2-(dimethylaminomethyl)-1,4-diazabicyclo[2.2.2]octane, 2-(dimethylaminomethyl)-5-methyl-1,4-diazabicyclo[2.2.2]octane, 2-(dimethylaminomethyl)-6-methyl-1,4-diazabicyclo[2.2.2]octane, 2-(dimethylaminomethyl)-5,6-dimethyl-1,4-diazabicyclo[2.2.2]octane, 2-(dimethylaminoethoxy)-1,4-diazabicyclo[2.2.2]octane, 2-(dimethylaminoethoxy)-5-methyl-1,4-diazabicyclo[2.2.2]octane, 2-(dimethylaminoethoxy)-6-methyl-1,4-diazabicyclo[2.2.2]octane, 2-(dimethylaminoethoxy)-5,6-dimethyl-1,4-diazabicyclo[2.2.2]octane, 2-[(dimethylaminoethoxy)carbonyl]-1,4-diazabicyclo[2.2.2]octane, 2-[(dimethylaminoethoxy)carbonyl]-5-methyl-1,4-diazabicyclo-[2.2.2]octane, 2-[(dimethylaminoethoxy)carbonyl]-6-methyl-1,4-diazabicyclo[2.2.2]-octane, 2-[(dimethylaminoethoxy)carbonyl]-5,6-dimethyl-1,4-diazabicyclo[2.2.2]octane, 2-(1′-hydroxypropyl)-1,4-diazabicyclo[2.2.2]octane, 2-(1′-hydroxypropyl)-5-methyl-1,4-diazabicyclo[2.2.2]octane, 2-(1′-hydroxypropyl)-6-methyl-1,4-diazabicyclo-[2.2.2]octane, 2-(1′-hydroxypropyl)-5,6-dimethyl-1,4-diazabicyclo[2.2.2]octane, 2-(2′-hydroxypropyl)-1,4-diazabicyclo[2.2.2]octane, 2-(2′-hydroxypropyl)-5-methyl-1,4-diazabicyclo[2.2.2]octane, 2-(2′-hydroxypropyl)-6-methyl-1,4-diazabicyclo-[2.2.2]octane, 2-(2′-hydroxypropyl)-5,6-dimethyl-1,4-diazabicyclo[2.2.2]octane, 2-(3′-hydroxypropyl)-1,4-diazabicyclo[2.2.2]octane, 2-(3′-hydroxypropyl)-5-methyl-1,4-diazabicyclo[2.2.2]octane, 2-(3′-hydroxypropyl)-6-methyl-1,4-diazabicyclo-[2.2.2]octane, 2-(3′-hydroxypropyl)-5,6-dimethyl-1,4-diazabicyclo[2.2.2]octane, 2,3-bis-(hydroxymethyl)-1,4-diazabicyclo[2.2.2]octane, 2,3-bis(hydroxymethyl)-5-methyl-1,4-diazabicyclo[2.2.2]octane, 2,3-bis(hydroxymethyl)-6-methyl-1,4-diazabicyclo-[2.2.2]octane, 2,3-bis(hydroxymethyl)-5,6-dimethyl-1,4-diazabicyclo[2.2.2]octane, 2,3-bis(ethoxycarbonyl)-1,4-diazabicyclo[2.2.2]octane, 2,3-bis(ethoxycarbonyl)-5-methyl-1,4-diazabicyclo[2.2.2]octane, 2,3-bis(ethoxycarbonyl)-6-methyl-1,4-diazabicyclo[2.2.2]octane and 2,3-bis(ethoxycarbonyl)-5,6-dimethyl-1,4-diazabicyclo-[2.2.2]octane.

In a further embodiment of the present invention, the dihydropyrazine (II) is prepared by reacting a dicarbonyl compound with ethylenediamine (EDA) or an EDA derivative. The carbonyl compound is preferably a diketo compound. Preferred diketo compounds are selected from 2,3-pentanedione, 2,3-butanedione, glyoxal, methylglyoxal. Preferred EDA derivatives are EDA, 1,2-propanediamine, 1,2-butanediamine, 2,3-butanediamine, 1,2-pentanediamine, 2,3-pentanediamine, 1,2-hexanediamine, 2,3-hexanediamine, 3,4-hexanediamine. Particular preference is given to the reactions of butanedione with ethylenediamine to give 2,3-dimethyl-5,6-dihydropyrazine, of methylglyoxal with ethylenediamine to give 2-methyl-5,6-dihydropyrazine, and of glyoxal and ethylenediamine to give 2,3-dihydropyrazine. The reaction is carried out in organic solvents, for example ethers, esters, alcohols or alkanes. Preference is given to performing the reaction in a moderately polar but water-immiscible solvent. Particular preference is given to using tert-butyl methyl ether (MTBE) as the solvent and additionally to working under N₂ as a protective gas.

The synthesis of the dihydropyrazines can be carried out between −80° C. and 80° C. Preference is given to carrying out the reaction between −20° C. and 60° C., particular preference to carrying it out between 0° C. and 50° C.

The reaction pressure may be between 0.5 and 250 bar (abs.). Preference is given to working at standard pressure.

The present invention further provides novel triethylenediamine derivatives which can be prepared by the process according to the invention. These inventive TEDA derivatives comprise substituents with functional groups, especially substituents which have at least one heteroatom such as halogen, O, P, S or N, preferably O or N. As already stated above, a few TEDA derivatives are already known. These already known TEDA derivatives do not form part of the subject matter of the present invention with regard to the TEDA derivatives as such. The inventive TEDA derivatives as such thus do not comprise the TEDA derivatives described in the aforementioned documents by T. Oishi et al., L. Street et al., E. Shiskhin et al. and in WO 98/24790 and DE-A 30 48 031.

The inventive TEDA derivatives correspond to the general formula (Ia) or (Ib)

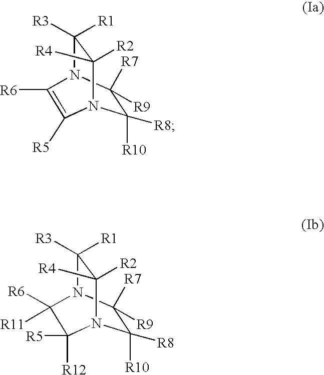

preparable by the process according to the invention, where the R1 to R12 radicals are each as defined above and where at least one of the substituents R1 to R10 comprises at least one heteroatom selected from halogen, O, P, S and N, preferably O and N, or at least one of the R1 to R10 radicals comprises —OH or —NH₂,
with the prerequisite that one of the R1 to R12 radicals is not —C(O)OH, —C(O)OCH₃, —C(O)OC₂H₅, —CH₂—OH, —CH₂—O-benzyl or —CH₂—O—C(O)—CH₃ when the other R1 to R12 radicals are each hydrogen, and two adjacent R1 to R12 radicals are not both —CH₂—O-benzyl when the other R1 to R12 radicals are each hydrogen. “Adjacent radical” is understood to mean that the particular radicals are bonded to 2 different carbon atoms of the inventive TEDA derivatives and these two carbon atoms are themselves in turn bonded to one another.

With regard to the preferred and particularly preferred inventive TEDA derivatives, reference is made to the corresponding definitions of the process according to the invention taking account of the above restrictions with regard to the TEDA derivatives as such.

The present invention further provides for the use of the inventive TEDA derivatives for preparing polyurethanes. Preference is given to using the inventive TEDA derivatives as a catalyst, especially in the production of polyurethane foams. Such processes for preparing polyurethanes are known to those skilled in the art. With regard to the selection of the reactants used in the polyurethane preparation, there are in principle no restrictions. The present invention therefore also provides polyurethanes comprising at least one inventive TEDA derivative. Such polyurethanes, preferably polyurethane foams, are notable in that the TEDA derivatives used do not outgas because they are chemically incorporated into the corresponding polyurethane. In this way, it is possible to prepare low-odor or odorless polyurethanes.

The inventive TEDA derivative can preferably be used, in its property as a polyurethane catalyst, as the gel catalyst for the crosslinking reaction or as a blowing catalyst for the release of CO₂ with the aid of water. Particular preference is given to using the inventive TEDA derivative as the gel catalyst which promotes the crosslinking reaction between polyisocyanate and polyol component.

The invention will be illustrated in detail with reference to the examples which follow:

### EXAMPLES

### Examples 1-12

### Preparation of 2,3-dimethyl-5,6-dihydropyrazine

### Example 1

1 g of 2,3-butanedione is initially charged in substance. 4 ml of a solution, made up beforehand, of 10 g of ethylenediamine in 30 g of H₂O are metered in dropwise; the reaction is exothermic. The reaction solution changes color during the reaction from yellow to dark brown. 2,3-Butanedione is initially charged pure, since an emulsion forms in water. GC analysis: [GC area %] 10.28% EDA, 76.94% 2,3-dimethyl-5,6-dihydropyrazine, 12.78% others.

### Example 2

Butanedione (52.9 g, 615 mmol, 1 eq.) is initially charged in 1,2-propanediol (50 g), and EDA (73.8 g, 1.23 mol, 2 eq.) is added with ice cooling. The temperature rises to approx. 40° C. The solution changes color through yellow to black. GC analysis on a 30 m RTX-5-Amine column shows that the product mixture consists of 14.7% EDA, 81.5% 1,2-propanediol and 0.61% 2,3-dimethyl-5,6-dihydropyrazine (calculated without solvent: 79.5% EDA, 3.29% 2,3-dimethyl-5,6-dihydropyrazine).

### Example 3

EDA (5.58 g, 92 mmol) is initially charged at 0° C. in 1,2-propanediol (20 ml), and butanedione (4.00 g, 46.4 mmol, 1 eq.) dissolved in 1,2-propanediol (20 ml) is added dropwise very slowly over 2.5 h, and the mixture is stirred vigorously. In the course of this, the temperature of the mixture does not rise above 5° C. In the meantime, a fine white solid precipitates out and dissolves slowly in the course of warming to room temperature. The slightly yellowish solution is stored at −20° C. GC analysis on a 30 m RTX-5-Amine column shows that the product mixture consists of 5.83% EDA, 79.4% 1,2-propanediol and 13.6% 2,3-dimethyl-5,6-dihydropyrazine (calculated without solvent: 28.2% EDA, 66.0% 2,3-dimethyl-5,6-dihydropyrazine).

### Example 4

EDA (5.58 g, 92 mmol) is initially charged at 0° C. in MeOH (20 ml). Butanedione (4.00 g, 46.4 mmol, 1 eq.) is dissolved in MeOH (40 ml) and added dropwise very slowly to the EDA solution over 2 h, and the mixture is stirred vigorously. In the course of this, the temperature of the mixture does not rise above 0° C. The white solid formed is filtered off and washed with cold MeOH. As the solvent is being drawn off at 30° C., the solid is converted to 2,3-dimethyl-5,6-dihydropyrazine (brown oil). GC analysis on a 30 m RTX-5-Amine column shows that the product mixture consists of 74.2% MeOH, 7.12% EDA and 18.2% 2,3-dimethyl-5,6-dihydropyrazine (calculated without solvent: 27.6% EDA, 70.5% 2,3-dimethyl-5,6-dihydropyrazine).

### Example 5

EDA (16.7 g, 278 mmol, 2 eq.) is initially charged at 0° C. in 1,2-propanediol (150 ml). Butanedione (12.0 g, 139 mmol) in 1,2-propanediol (60 ml) is added dropwise (over 1.5 h) and stirred vigorously at the same time. The solution is stirred at 0° C. for a further 30 min and stored at −20° C. overnight. GC analysis on a 30 m RTX-5-Amine column shows that the product mixture consists of 3.66% EDA, 86.0% 1,2-propanediol and 9.90% 2,3-dimethyl-5,6-dihydropyrazine (calculated without solvent: 26.1% EDA, 70.7% 2,3-dimethyl-5,6-dihydropyrazine).

### Example 6

Operation is effected under N₂. EDA (2.79 g, 46.4 mmol, 2 eq.) is initially charged at 0° C. in MTBE (60 ml). Butanedione (4.00 g, 46.4 mmol) in MTBE (6 ml) is added dropwise (over 40 min). As a result of the white precipitate, the mixture becomes very thick and is therefore stirred vigorously. It is subsequently heated rapidly to 50° C. in order to dissolve the precipitate. Two phases form. Immediately thereafter, the mixture is cooled to 0° C. in order to avoid polymerization. The organic phase is fractionally distilled. The desired product boils at 28 mbar/95° C. and is collected in a cold trap. GC analysis of the phases shows that the ethereal phase comprises virtually only product, while the aqueous phase comprises all impurities and a portion of the product. The product can be extracted by shaking with tert-butyl methyl ether (MTBE) without entraining the by-products. GC analysis on a 30 m RTX-5-Amine column shows that the organic phase consists of 0.03% EDA, 88.2% MTBE and 11.5% 2,3-dimethyl-5,6-dihydropyrazine (calculated without solvent: 0.25% EDA, 97.5% 2,3-dimethyl-5,6-dihydropyrazine).

### Example 7

EDA (13.9 g, 232 mmol, 1 eq.) is initially charged at 0° C. in MTBE (125 ml). Butanedione (20.0 g, 232 mmol) in MTBE (30 ml) is added dropwise (over 1.5 h). The mixture is subsequently stirred at 0° C. for 15 min, then warmed to ambient temperature and stirred until two phases form. The phases which form are separated and the aqueous phase is extracted by shaking with MTBE (3×25 ml). The ethereal phases are combined and dried over MgSO₄. The ether is removed at 50° C./613 mbar. The product is obtained at 7 mbar/105° C. (bottom)/40° C. (top). GC analysis on a 30 m RTX-5-Amine column shows that the product mixture consists of 83.1% MTBE, 16.5% 2,3-dimethyl-5,6-dihydropyrazine and 0.44% others (calculated without solvent: 97.6% 2,3-dimethyl-5,6-dihydropyrazine, 2.60% others).

### Example 8

EDA (25.51 g, 425 mmol, 1 eq.) is initially charged at 0° C. in MTBE (130 ml). Butanedione (36.56 g, 425 mmol) in MTBE (20 ml) is added dropwise (over 2 h). The mixture is stored at −20° C. for 50 h, then heated to 40° C., and the aqueous phase is removed. The aqueous phase is extracted by shaking with MTBE (3×25 ml). The ethereal phases are combined and dried over MgSO₄. The ether is removed at 30° C./395 mbar and the residue is fractionally distilled (spinning band column).

### Example 9

EDA (20.0 g, 232 mmol, 1 eq.) is initially charged at 0° C. in MTBE (125 ml). Butanedione (13.9 g, 232 mmol) in MTBE (30 ml) is added dropwise (over 1.5 h). The mixture is heated to 35° C. and the aqueous phase is removed. The aqueous phase is extracted by shaking with MTBE (3×15 ml). The ethereal phases are combined and dried over MgSO₄. The ether is removed at 33° C./410 mbar. The residue is combined with the residue from Experiment 15 and fractionally distilled. The product is obtained at 43 mbar/100° C. (bottom)/72.2° C. (top). The isolated molar yield is 66.2%, the purity of the distilled product by GC 99.1%.

### Example 10

The EDA (60 g, 1 mol, 1 eq.) is initially charged in 350 ml of MTBE, and the butanedione (86 g, 1 mol, 1 eq.) is added dropwise at 0° C. The mixture becomes solid in places and warms up briefly to 35° C. 50 ml of MTBE are added and the abatement of the reaction is awaited. The solution is stored at −20° C. for 55 h, then heated to 40° C., and the two phases are separated.

### Example 11

Butanedione (172 g, 2 mol, 1 eq.) is initially charged in MTBE, and EDA (120 g, 2 mol, 1 eq. in 120 ml of MTBE) is slowly added dropwise. The mixture is heated to 30° C. and the aqueous phase is extracted with 3×100 ml of MTBE. The combined organic phases are dried with MgSO₄ and MTBE is removed as far as possible at 55° C. on a rotary evaporator. The residue is fractionally distilled by means of a spinning band column. During the dropwise addition, the temperature in the flask rises to approx. 35° C., since the reaction mixture solidifies in places. The dropwise addition is stopped immediately and the abatement of the reaction is awaited. The solution does not darken in color. Brief heating above 30° C. does not appear to have have any further effects on the course of the reaction.

### Example 12

EDA (240 g, 4 mol, 1 eq.) is initially charged in 750 ml of MTBE, and butanedione (320 g, 3.72 mol, 0.93 eq.) dissolved in 200 ml of MTBE is added dropwise at 10° C. The mixture is stirred at ambient temperature overnight (14 h). The phases are separated, the aqueous layer is extracted by shaking with 3×50 ml of MTBE and the combined organic phases are dried with MgSO₄. The ether is removed on a rotary evaporator at 40° C. The crude product is not worked up any further since the MTBE still present does not disrupt other reactions. GC analysis on a 30 m RTX-5-Amine column shows that the product mixture consists of 25.6% MTBE, 0.14% EDA, 71.9% 2,3-dimethyl-5,6-dihydropyrazine and 2.36% others (calculated without solvent: 0.19% EDA, 96.6% 2,3-dimethyl-5,6-dihydropyrazine, 3.17% others).

### Examples 13-17

### Preparation of 2,3-dihydropyrazine

2,3-Dihydropyrazine shows a strong tendency to dimerize and polymerize, which is why it is preferable to work at low temperatures. After the synthesis of the dihydropyrazine, the subsequent stage should be begun as soon as possible.

### Example 13

The ethylenediamine (6.0 g, 0.1 mol, 1 eq.) is dissolved in MTBE (40 ml) and cooled to 0° C. Glyoxal (5.8 g, 0.1 mol, 1 eq., 14.5 g of 40% solution in H₂O) is added dropwise at such a rate that the temperature does not rise above 0° C. The evolution of heat in the reaction is much less than when methylglyoxal or butanedione is used. The aqueous phase is virtually colorless. The desired product is detected by GC-MS but not isolated. After 1 h at ambient temperature, the residue solidifies to a rubberlike substance.

### Example 14

30 g [0.5 mol] of ethylenediamine and 30 g [1.67 mol] of water are initially charged. 18.2 g of 40% aqueous glyoxal solution [0.125 mol of glyoxal] are then added dropwise to the warm solution. An exothermic reaction proceeds. GC analysis: 75.53% EDA, 15.36% 2,3-dihydropyrazine, 9% unknown (52.69% H₂O).

### Example 15

A test tube is initially charged with 50% EDA solution in water, and glyoxal (40% solution in water) is added dropwise. An exothermic reaction takes place. The gas chromatogram of the reaction mixture (30 m RTX 5 amine) shows the following composition (in GC area %):

50.93% ethylenediamine, 2.11% monoethylene glycol, 26.11% 2,3-dihydropyrazine, 20.85% others. The desired product is confirmed by GC-MS.

### Example 16

A 250 ml flask with magnetic stirrer, dropping funnel and mini-separating column is initially charged with 60 g [1 mol] of ethylenediamine and 60 g [3.33 mol] of water. Once the mixture (heating to 50° C. as a result of heat of mixing) has cooled to 38° C., 36.3 g of a 40% aqueous glyoxal solution [0.25 mol of glyoxal] are added dropwise within 15 minutes, in the course of which the mixture warms up to 62° C. The gas chromatogram of the reaction mixture (30 m RTX 5 amine) shows the following composition (in GC area %): 74.5% ethylenediamine, 14.6% 2,3-dihydropyrazine, 10.9% unknown compounds, water content not included.

### Example 17

30 g [0.5 mol] of ethylenediamine and 30 g [1.67 mol] of water are initially charged. 18.2 g of 40% aqueous glyoxal solution [0.125 mol of glyoxal] are then added dropwise to the warm EDA solution. The gas chromatogram of the reaction mixture (30 m RTX 5 amine) shows the following composition (in GC area %):

77.3% ethylenediamine, 12.7% 2,3-dihydropyrazine, 10.0% unknown compounds, water content not included.

### Examples 18-20

### Preparation of Further Dihydropyrazines

### Example 18

### 2-Methyl-5,6-dihydropyrazine

EDA (6.0 g, 0.1 mol, 1 eq.) is dissolved in MTBE (40 ml) and cooled to 0° C. Methylglyoxal (7.2 g, 0.1 mol, 1 eq., 18 g of 40% solution in H₂O) is added dropwise at such a rate that the temperature does not rise above 5° C. The solution turns yellowish. After warming to ambient temperature, the phases are separated and the aqueous phase takes on a black color. For a GC sample, a portion of the aqueous phase is diluted to a high degree with acetone. The desired product is detected by GC-MS but not isolated.

### Example 19

### 2-Methyl-5,6-dihydropyrazine

The EDA (4.5 g, 75 mmol, 3 eq.) is dissolved in MTBE (40 ml) and cooled to 0° C., and methylglyoxal (1.8 g, 25 mmol, 1 eq., 4.5 g of 40% solution in H₂O) is added dropwise (30 min). The mixture is warmed to ambient temperature overnight. The organic phase is colorless; the aqueous phase has a light brown color. The aqueous phase comprises principally 3 main products. The desired product is detected in both phases by GC-MS, but not isolated.

### Example 20

### 2-Ethyl-3-methyl-5,6-dihydropyrazine

To a solution consisting of one part of ethylenediamine in one part of 1,2-propanediol is slowly added a solution of one part of 2,3-pentanedione dissolved in one part of 1,2-propanediol. An exothermic reaction takes place. The gas chromatogram of the reaction mixture (30 m RTX 5 amine) shows the following composition (in GC area %): 25.28% ethylenediamine, 56.76% 1,2-propanediol, 16.62% 2-ethyl-3-methyl-5,6-dihydropyrazine, 0.77% others.

Calculated without solvent: 58.46% ethylenediamine, 38.42% 2-ethyl-3-methyl-5,6-dihydropyrazine, 3.12% others. The desired product is confirmed by GC-MS.

### Preparation of the TEDA Derivatives (Ia)

### Examples 21-33

### 2-Ethoxycarbonyl-5,6-dimethyl-1,4-diazabicyclo[2.2.2]oct-5-ene

### Example 21

A portion of the effluent from Example 1 (77% 2,3-dimethyl-5,6-dihydropyrazine) is initially charged; 1 g of ethyl acrylate is added. At room temperature, there is no reaction. The reaction vessel is heated to 100° C. for a few minutes. The GC analysis (30 m RTX-5 amine) of the reaction effluent shows the following composition:

0.17% EDA, 4.24% ethyl acrylate,
46.91% 2,3-dimethyl-5,6-dihydropyrazine, 1.03% N-acetyl-EDA,
10.67% 2-ethoxycarbonyl-5,6-dimethyl-1,4-diazabicyclo[2.2.2]oct-5-ene (product),
17.51% N,N-bis[2′-(ethoxycarbonyl)ethyl]ethylenediamine, 18.09% others.

### Example 22

2,3-Dimethyl-5,6-dihydropyrazine from Example 4 (approx. 23 mmol, dissolved in 40 ml of MeOH, 1 eq.) and ethyl acrylate (2.50 g, 25 mmol, 1.1 eq.) are heated to 65° C. (reflux) under N₂. After 30 min, 1 h, 1.5 h, 2 h and 3 h, a sample is taken for GC in order to monitor the course of the reaction. After 30 min, the gas chromatogram of the reaction mixture (30 m RTX 5 amine) shows the following composition (in GC area %):

74.8% methanol, 1.43% ethylenediamine, 9.80% 2,3-dimethyl-5,6-dihydropyrazine, 2.31% 2-hydroxyethylpiperazine, 3.04% N-acetyl-EDA, 1.93% N,N-bis[2′-(ethoxycarbonyl)ethyl]ethylenediamine, 6.69% others.

After 90 min, the gas chromatogram of the reaction mixture (30 m RTX 5 amine) shows the following composition (in GC area %):

69.9% methanol, 1.59% ethylenediamine, 11.3% 2,3-dimethyl-5,6-dihydropyrazine, 3.79% 2-hydroxyethylpiperazine, 2.23% N-acetyl-EDA, 0.71% N,N-bis[2′-(ethoxycarbonyl)ethyl]ethylenediamine, 10.5% others.

After 180 min, the gas chromatogram of the reaction mixture (30 m RTX 5 amine) shows the following composition (in GC area %):

68.0% methanol, 1.49% ethylenediamine, 11.6% 2,3-dimethyl-5,6-dihydropyrazine, 4.21% 2-hydroxyethylpiperazine, 1.08% N-acetyl-EDA,
0.17% N,N-bis[2′-(ethoxycarbonyl)ethyl]ethylenediamine, 13.5% others.

Calculated without solvent: 4.66% ethylenediamine, 36.3% 2,3-dimethyl-5,6-dihydropyrazine, 13.2% 2-hydroxyethylpiperazine, 3.38% N-acetyl-EDA, 0.53% N,N-bis[2′-(ethoxycarbonyl)ethyl]ethylenediamine, 42.2% others.

### Example 23

2,3-Dimethyl-5,6-dihydropyrazine from Example 3 (approx. 23 mmol in 40 ml of 1,2-propanediol, 1 eq.) and ethyl acrylate (2.50 g, 25 mmol, 1.1 eq.) are heated to 100° C. under N₂. After 30 min, 1 h and 1.5 h, a sample is taken for GC in order to monitor the course of the reaction. After 30 min, the gas chromatogram of the reaction mixture (30 m RTX 5 amine) shows the following composition (in GC area %): 0.94% ethylenediamine, 81.5% 1,2-propanediol, 7.10% 2,3-dimethyl-5,6-dihydropyrazine, 1.18% N-acetyl-EDA, 0.08% 2-ethoxycarbonyl-5,6-dimethyl-1,4-diazabicyclo-[2.2.2]oct-5-ene (product), 9.20% others.

At 90 min, the gas chromatogram of the reaction mixture (30 m RTX 5 amine) shows the following composition (in GC area %):

1.28% ethylenediamine, 84.9% 1,2-propanediol, 0.25% piperazine, 4.12% 2,3-dimethyl-5,6-dihydropyrazine, 0.31% N-acetyl-EDA, 0.04% 2-ethoxycarbonyl-5,6-dimethyl-1,4-diazabicyclo[2.2.2]oct-5-ene (product), 9.10% others.

Calculated without solvent: 8.48% ethylenediamine, 1.66% piperazine, 27.3% 2,3-dimethyl-5,6-dihydropyrazine, 2.05% N-acetyl-EDA, 0.26% 2-ethoxycarbonyl-5,6-dimethyl-1,4-diazabicyclo[2.2.2]oct-5-ene (product), 60.36% others.

### Example 24

2,3-Dimethyl-5,6-dihydropyrazine (approx. 12 mmol in 20 ml of 1,2-propanediol, 1 eq.) and 1,2-propanediol (30 ml) are heated to 60° C. At this temperature, ethyl acrylate (6.0 g, 60 mmol, 0.5 eq. dissolves in 2.0 ml of 1,2-propanediol) is added dropwise under N₂ (10 min), and the mixture is then heated rapidly to 100° C. The reaction solution is left at this temperature for 5 min. The gas chromatogram of the reaction mixture (30 m RTX 5 amine) shows the following composition (in GC area %):

91.0% 1,2-propanediol, 1.67% 2,3-dimethyl-5,6-dihydropyrazine, 0.03% N-acetyl-EDA, 1.06% 2-ethoxycarbonyl-5,6-dimethyl-1,4-diazabicyclo[2.2.2]oct-5-ene (product), 6.24% others.

Calculated without solvent: 18.6% 2,3-dimethyl-5,6-dihydropyrazine, 0.33% N-acetyl-EDA, 11.8% 2-ethoxycarbonyl-5,6-dimethyl-1,4-diazabicyclo[2.2.2]oct-5-ene (product), 69.3% others.

### Example 25

2,3-Dimethyl-5,6-dihydropyrazine (approx. 15 mmol in 20 ml of 1,2-propanediol, 1 eq.) and ethyl acrylate (15 g, 0.15 mmol, 10 eq.) are heated to 100° C. under N₂. The reaction solution is left at this temperature for 22 min, in the course of which it turns red-brown. The yield cannot be optimized by a massive excess of acrylic ester.

### Example 26

2,3-Dimethyl-5,6-dihydropyrazine is purified by distillation before the experiment (Example 9). The high-boiling, substantially inert solvent selected is dioxane. The acrylic ester is used only in a small excess (1.2 equivalents). 2,3-Dimethyl-5,6-dihydropyrazine (1.778 g, 16.16 mmol, 1 eq.) is dissolved under N₂ in dioxane (25 ml) and heated to 80° C. Ethyl acrylate (1.939 g, 19.39 mmol, 1.2 eq.) is dissolved in dioxane (8 ml) and slowly added dropwise (15 min). The mixture is heated to reflux at 95° C. for 4 h, left to stand at 20° C. overnight and heated for another 4 h. The yield of desired product can be enhanced significantly.

The gas chromatogram of the reaction mixture (30 m RTX 5 amine) shows the following composition: (in GC area %.)

95.2% dioxane, 0.61% 2,3-dimethyl-5,6-dihydropyrazine, 1.26% 2-ethoxycarbonyl-5,6-dimethyl-1,4-diazabicyclo[2.2.2]oct-5-ene (product), 0.64%+0.35% addition products of the main product plus further equivalents of acrylic ester, 1.94% others.

Calculated without solvent: 12.7% 2,3-dimethyl-5,6-dihydropyrazine, 26.3% 2-ethoxycarbonyl-5,6-dimethyl-1,4-diazabicyclo[2.2.2]oct-5-ene (product), 13.3%+7.29% addition products of the main product plus further equivalents of acrylic ester, 40.4% others.

### Example 27

EDA (6.0 g, 0.1 mol, 1 eq.) is initially charged at 0° C. in dioxane (10 g), and butanedione (8.6 g, 0.1 mol, 1 eq.) in dioxane (2.0 g) is slowly added dropwise. 12 g of dioxane are added and the mixture is stirred for a further 15 min. The apparatus is inertized with N₂ and admixed with ethyl acrylate (10 g, 0.1 mol, 1 eq.). The solution is heated to boiling (95° C.) and left at this temperature for 3 h. The desired product can be detected by GC-MS.

### Example 28

Distilled 2,3-dimethyl-5,6-dihydropyrazine (11 g, 0.1 mol, 1 eq., Example 9) is heated to 80° C. in dioxane (75 ml) under N₂, and ethyl acrylate (12 g, 0.12 mol, 1.2 eq.) is added. The mixture is heated to reflux (97° C.) for 3.5 h, cooled to −20° C. for 14 h and heated again to reflux for 2 h. The gas chromatogram of the reaction mixture (30 m RTX 5 amine) shows the following composition (in GC area %):

65.8% dioxane, 11.1% 2,3-dimethyl-5,6-dihydropyrazine, 10.1% 2-ethoxycarbonyl-5,6-dimethyl-1,4-diazabicyclo[2.2.2]oct-5-ene (product), 13.0% others.

Calculated without solvent: 32.5% 2,3-dimethyl-5,6-dihydropyrazine, 29.5% 2-ethoxycarbonyl-5,6-dimethyl-1,4-diazabicyclo[2.2.2]oct-5-ene (product), 38.0% others.

### Example 29

Distilled 2,3-dimethyl-5,6-dihydropyrazine (34.3 g, 0.321 mol, 1 eq., Example 9) is heated to 88° C. in dioxane (50 ml) under N₂, and to this is added ethyl acrylate (44.9 g, 0.499 mol, 1.55 eq.). The mixture is heated to 105° C. for 4 h, cooled to −20° C. for 14 h and heated again to 105° C. for 2 h. The gas chromatogram of the reaction mixture (30 m RTX 5 amine) shows the following composition (in GC area %):

34.1% dioxane, 11.5% 2,3-dimethyl-5,6-dihydropyrazine, 27.0% 2-ethoxycarbonyl-5,6-dimethyl-1,4-diazabicyclo[2.2.2]oct-5-ene (product), 5.40%+12.2% addition products of the main product plus further equivalents of acrylic ester, 9.80% others.

Calculated without solvent: 17.5% 2,3-dimethyl-5,6-dihydropyrazine, 41.0% 2-ethoxycarbonyl-5,6-dimethyl-1,4-diazabicyclo[2.2.2]oct-5-ene (product), 8.19%+18.5% addition products of the main product plus further equivalents of acrylic ester, 14.9% others. This result corresponds to a conversion of 59.5% based on dimethyldihydropyrazine and a selectivity of the dimethyldihydropyrazine for the desired product of 79.0%.

### Example 30

2,3-Dimethyl-5,6-dihydropyrazine (40.3 g, 0.367 mol, 1 eq., Example 9) and ethyl acrylate (39.6 g, 0.396 mol, 1.2 eq.) are heated to 98° C. in dioxane (100 ml) under N₂ and stirred for 4.5 h. The mixture is stored at −20° C. overnight (14 h). The crystals formed are analyzed by means of GC; they have the same composition as the reaction solution. The reaction is continued at 93° C. for 8 h. The gas chromatogram of the reaction mixture (30 m RTX 5 amine) shows the following composition (in GC area %): 1.34% ethyl acrylate, 40.8% dioxane, 14.8% 2,3-dimethyl-5,6-dihydropyrazine, 28.4% 2-ethoxycarbonyl-5,6-dimethyl-1,4-diazabicyclo[2.2.2]oct-5-ene (product), 4.50%+9.75% addition products of the main product plus further equivalents of acrylic ester, 0.41% others.

Calculated without solvent: 2.26% ethyl acrylate, 25.0% 2,3-dimethyl-5,6-dihydropyrazine, 48.0% 2-ethoxycarbonyl-5,6-dimethyl-1,4-diazabicyclo[2.2.2]oct-5-ene (product), 7.60%+16.5% addition products of the main product plus further equivalents of acrylic ester, 0.69% others. This result corresponds to a conversion of 50.3% based on dimethyldihydropyrazine or 95.4% based on ethyl acrylate, and a selectivity of the dimethyldihydropyrazine for the desired product of approx. 90%.

### Example 31

2,3-Dimethyl-5,6-dihydropyrazine (11 g, 0.1 mol, 1 eq.) and ethyl acrylate (10 g, 0.1 mol, 1 eq.) are dissolved in 20 ml of THF and heated to 99° C. under 80 bar of H₂. Further H₂ is injected up to 200 bar and the mixture is stirred for a total of 7 h. The gas chromatogram of the reaction mixture (30 m RTX 5 amine) shows the following composition (in GC area %):

48.7% THF, 1.04% ethyl acrylate, 10.6% 2,3-dimethyl-5,6-dihydropyrazine, 23.0% 2-ethoxycarbonyl-5,6-dimethyl-1,4-diazabicyclo[2.2.2]oct-5-ene (product), 5.66%+9.70% addition products of the main product plus further equivalents of acrylic ester, 1.30% others.

Calculated without solvent: 2.03% ethyl acrylate, 20.7% 2,3-dimethyl-5,6-dihydropyrazine, 44.8% 2-ethoxycarbonyl-5,6-dimethyl-1,4-diazabicyclo[2.2.2]oct-5-ene (product), 11.0%+18.9% addition products of the main product plus further equivalents of acrylic ester, 2.53% others. This result corresponds to a conversion of 60.5% based on dimethyldihydropyrazine or 95.7% based on ethyl acrylate and a selectivity of the dimethyldihydropyrazine for the desired product of 74%.

### Example 32

2,3-Dimethyldihydropyrazine (141 g, 1.28 mol, 1 eq., 196 g of an approx. 72% solution in MTBE) and ethyl acrylate (128 g, 1.28 mol, 1 eq.) are heated together to 82° C. and stirred at this temperature for a total of 11.5 h until no acrylic ester is detectable any longer in the GC. The gas chromatogram of the reaction mixture (30 m RTX 5 amine) shows the following composition (in GC area %):

7.70% MTBE, 16.9% 2,3-dimethyl-5,6-dihydropyrazine 46.2% 2-ethoxycarbonyl-5,6-dimethyl-1,4-diazabicyclo[2.2.2]oct-5-ene (product), 8.92%+17.3% addition products of the main product plus further equivalents of acrylic ester, 2.98% others.

Calculated without solvent: 18.3% 2,3-dimethyl-5,6-dihydropyrazine, 50.1% 2-ethoxycarbonyl-5,6-dimethyl-1,4-diazabicyclo[2.2.2]oct-5-ene (product), 9.67%+18.7% addition products of the main product plus further equivalents of acrylic ester, 3.23% others. This result corresponds to a conversion of 65.1% based on dimethyldihydropyrazine or 100% based on ethyl acrylate, and a selectivity of the dimethyldihydropyrazine for the desired product of 76.1%.

### Example 33

2,3-Dimethyldihydropyrazine (112 g, 1.02 mol, 1 eq., 156 g of an approx. 72% in MTBE) and ethyl acrylate (102 g, 1.02 mol, 1 eq.) are heated together to 82° C. and stirred at this temperature for a total of 6 h. The gas chromatogram of the reaction mixture (30 m RTX 5 amine) shows the following composition (in GC area %):

19.4% MTBE, 8.10% ethyl acrylate, 20.0% 2,3-dimethyl-5,6-dihydropyrazine, 34.3% 2-ethoxycarbonyl-5,6-dimethyl-1,4-diazabicyclo[2.2.2]oct-5-ene (product), 6.28%+10.1% addition products of the main product plus further equivalents of acrylic ester, 1.82% others.

Calculated without solvent: 10.0% ethyl acrylate, 24.8% 2,3-dimethyl-5,6-dihydropyrazine, 42.6% 2-ethoxycarbonyl-5,6-dimethyl-1,4-diazabicyclo[2.2.2]oct-5-ene (product), 7.79%+12.5% addition products of the main product plus further equivalents of acrylic ester, 2.26% others. This result corresponds to a conversion of 52.6% based on dimethyldihydropyrazine or 79% based on ethyl acrylate and a selectivity of the dimethyldihydropyrazine for the desired product of 81.0%.

### Examples 34-35

2,3-Bis(ethoxycarbonyl)-5,6-dimethyl-1,4-diazabicyclo[2.2.2]oct-5-ene

### Example 34

The experiment is carried out under N₂ as a protective gas. 2,3-Dimethyldihydropyrazine (11 g, 0.1 mol, 1 eq.) is initially charged and ⅓ of the amount of dimethyl maleate (14.4 g, 0.1 mol, 1 eq.) is added dropwise. Slight turbidity occurs immediately, then the mixture becomes reddish in color. The remaining ester is added and the mixture is stirred for 3 h. The mixture is then heated to 85° C. for 1 h. The mixture becomes very viscous; in the course of cooling, crystals form in the neck of the flask and the contents solidify. A sample is diluted with acetone and analyzed by gas chromatography. The gas chromatogram of the reaction mixture (30 m RTX 5 amine) shows the following composition (in GC area %):

3.40% MeOH, 19.1% acetone, 8.46% 2,3-dimethyl-5,6-dihydropyrazine, 1.51% diethyl maleate, 40.26% 2,3-bis(ethoxycarbonyl)-5,6-dimethyl-1,4-diazabicyclo[2.2.2]oct-5-ene (product), 19.5% others.

Calculated without solvent: 10.95% 2,3-dimethyl-5,6-dihydropyrazine, 1.95% diethyl maleate, 52.13% 2,3-bis(ethoxycarbonyl)-5,6-dimethyl-1,4-diazabicyclo[2.2.2]oct-5-ene (product), 25.25% others.

This result corresponds to a conversion of 74.7% based on dimethyldihydropyrazine or 96.5% based on diethyl maleate, and a selectivity of the dimethyldihydropyrazine for the desired product of 63.3%.

### Example 35

2,3-Dimethyldihydropyrazine (5.5 g, 50 mmol, 1 eq.) is dissolved in MTBE (30 ml) and cooled to approx. −30° C. (ice, dry ice and NaCl). Dimethyl maleate (7.2 g, 50 mmol, 1 eq.) is slowly added dropwise such that the temperature does not change significantly. The mixture is stirred overnight and, in the course of this, warmed slowly to ambient temperature. It is left to stand at ambient temperature for 10 days, in the course of which the contents turn increasingly dark brown but do not become viscous as in Example 31. The gas chromatogram of the reaction mixture (30 m RTX 5 amine) shows the following composition (in GC area %):

0.31% MeOH, 70.1% MTBE, 3.84% 2,3-dimethyl-5,6-dihydropyrazine, 11.1% diethyl maleate, 11.6% 2,3-bis(ethoxycarbonyl)-5,6-dimethyl-1,4-diazabicyclo[2.2.2]oct-5-ene (product), 3.05% others.

Calculated without solvent: 13.0% 2,3-dimethyl-5,6-dihydropyrazine, 37.5% diethyl maleate, 39.2% 2,3-bis(ethoxycarbonyl)-5,6-dimethyl-1,4-diazabicyclo[2.2.2]oct-5-ene (product), 10.3% others.

This result corresponds to a conversion of 70.0% based on dimethyldihydropyrazine or 33.9% based on diethyl maleate, and a selectivity of the dimethyldihydropyrazine for the desired product of 50.9%.

### Examples 36-38

### Further Diazabicyclooctenes

### 2-Hydroxymethyl-5,6-dimethyl-1,4-diazabicyclo[2.2.2]oct-5-ene

### Example 36

2,3-Dimethyldihydropyrazine (5.50 g, 50 mmol, 1 eq.) is dissolved in MTBE (30 ml), and allyl alcohol (5.00 g, 50 mmol, 5.39 ml, 1 eq.) is added. The mixture is boiled under reflux at 62° C. for 4 h, then stirred at ambient temperature for 65 h. A little product (2.83%) is formed, which was detected by GC-MS.

### 2-Ethoxycarbonyl-5-methyl-6-ethyl-1,4-diazabicyclo[2.2.2]oct-5-ene and 2-Ethoxycarbonyl-5-ethyl-6-methyl-1,4-diazabicyclo[2.2.2]oct-5-ene

### Example 37

To 2 parts of the reaction solution from Example 20 (2-ethyl-3-methyl-5,6-dihydropyrazine) is added 1 part of ethyl acrylate, and the mixture is heated to 100° C. for a few minutes. The gas chromatogram of the reaction mixture (30 m RTX 5 amine) shows the following composition (in GC area %):

3.63% ethanol, 1.53% ethyl acrylate, 43.92% 1,2-propanediol, 7.39% 2-ethyl-3-methyl-5,6-dihydropyrazine, 7.32% 2-ethoxycarbonyl-5-methyl-6-ethyl-1,4-diazabicyclo[2.2.2]-oct-5-ene, 36.21% others, in particular EDA+acrylic ester addition products. The identity of the product was confirmed by GC-MS; no statement can be made regarding the selectivity between the two possible reaction products 2-ethoxycarbonyl-5-methyl-6-ethyl-1,4-diazabicyclo[2.2.2]oct-5-ene and 2-ethoxycarbonyl-5-ethyl-6-methyl-1,4-diazabicyclo[2.2.2]oct-5-ene.

### Example 38

2,3-Dihydropyrazine from Example 16 is mixed with ethyl acrylate and heated to 80° C. for a few minutes. A little product (3.4%) is formed, which is detected by GC-MS. The reaction conditions are not yet optimized and accordingly not yet adjusted to the high reactivity of 2,3-dihydropyrazine.

### Preparation of the TEDA Derivatives (Ib)

The ester function and the double bond are hydrogenated by the methods known to those skilled in the art, for example with LiAlH₄ according to the literature description of G. Shishkin et al., Chem. Heterocycl. Com., 1980, pages 1069 to 1072, or with hydrogen over homogeneous or heterogeneous catalysts.

1.-10. (canceled)

11. A process for preparing triethylenediamine (TEDA) derivatives, comprising the following steps:
a) reacting a dihydropyrazine with an olefin, and
b) optionally hydrogenating after step a).

12. The process according to claim 11 for preparing triethylenediamine (TEDA) derivatives of the general formulae (Ia) and (Ib) 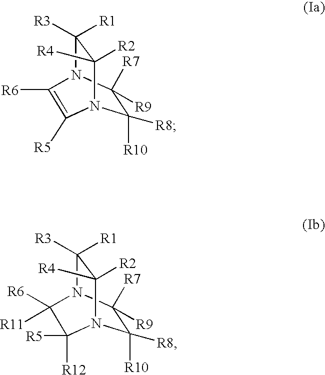
comprising the following steps:
a) reacting a dihydropyrazine (II) with an olefin (III) to obtain the TEDA derivative (Ia) 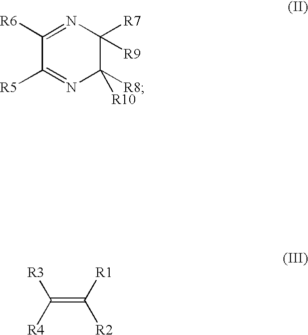
b) optionally hydrogenating the TEDA derivative (Ia) to obtain the TEDA derivative (Ib),
where, in the formulae (I) to (III):
R1 to R10 are each independently, halogen, —(C₁-C₁₀-alkyl)-(R14)ᵤ, —C(O)—R13, —CN, —O-aryl(R14)ᵤ, —(C₁-C₁₀-alkyl)-O—C(O)R14, [(C₁-C₁₀-alkyl)-(R14)ᵤ]ₚH_{q}, —N—[(C₁-C₁₀-alkyl)-(R14)ᵤ]ₚH_{q}Aᵣ, —P—[(C₁-C₁₀-alkyl)-(R14)ᵤ]ₚH_{q}Aᵣ or —S—[(C₁-C₁₀-alkyl)-(R14)ᵤ]ₚH_{q};
u is from 0 to 10;
R11 and R12 are each H;
R13 is H, hydroxyl, amino, aryl, C₁-C₁₀-alkoxy, amino(C₁-C₁₀-alkoxy), alkylamino-(C₁-C₁₀-alkoxy), dialkylamino(C₁-C₁₀-alkoxy), amino(C₁-C₁₀-alkyl), aminoaryl, —NH(C₁-C₁₀-alkyl), —N(C₁-C₁₀-alkyl)₂, —NH-aryl, —N-(aryl)₂, halogen, hydroxyaryl, —O-aryl or —O—(C₁-C₁₀-alkyl)aryl;
R14 is hydroxyl, amino, aryl, C₁-C₁₀-alkoxy, amino(C₁-C₁₀-alkoxy), alkylamino-(C₁-C₁₀-alkoxy), dialkylamino(C₁-C₁₀-alkoxy), amino(C₁-C₁₀-alkyl), —NH(C₁-C₁₀-alkyl), —N(C₁-C₁₀-alkyl)₂, hydroxyaryl, aminoaryl, —NH-aryl, —N(aryl)₂, halogen, —PH-aryl, —P(aryl)₂, —O-aryl or —O—(C₁-C₁₀-alkyl)aryl;
q is from 0 to 3,
r is 0 or 1,
p is from 0 to 3 in N- or P-containing substituents, where p+q=2 or 3,
p is 0 or 1 in O- or S-containing substitutents, where p+q=1, and
A is an anion.

13. The process according to claim 11, wherein at least one of the substituents R1 to R10 in the dihydropyrazine (I) or in the olefin (III) is selected from the group consisting of —[(C₁-C₁₀-alkyl)-(R14)ᵤ], —O—[(C₁-C₁₀-alkyl)-(R14)ᵤ], —N[(C₁-C₁₀-alkyl)-(R14)ᵤ]₂, —NH[(C₁-C₁₀-alkyl)-(R14)ᵤ]—C(O)—R13 and —CN,
where R13 and R14 are each independently hydroxyl, C₁-C₁₀-alkoxy, —NH₂, —NH(C₁-C₁₀-alkyl), —N(C₁-C₁₀-alkyl)₂, —O-aryl or —O—(C₁-C₁₀-alkyl)aryl, and
u is from 0 to 10.

14. The process according to claim 11, wherein the TEDA derivative (Ia) is a compound of the formula (Ia1) 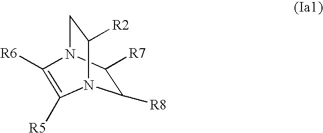
wherein R2, R7 and R8 are each independently H, —OH, —(C₁-C₃-alkyl)-OH, —(C₁-C₃-alkyl)-O—(C₁-C₃-alkyl), —CN, —O-phenyl, —(C₁-C₃-alkyl)-O-phenyl, C₁-C₃-alkoxy, —C(O)(C₁-C₃-alkoxy), —C(O)OH, —N(CH₃)₂, —NH(CH₃), —NH₂, —(C₁-C₃-alkyl)-N(CH₃)₂, —(C₁-C₃-alkyl)-NH(CH₃), —(C₁-C₃-alkyl)-NH₂, or —(C₁-C₃-alkyl)-O—C(O)(C₁-C₃-alkoxy), R5 and R6 are each independently H, C₁-C₃-alkyl, —C(O)OH, —C(O)(C₁-C₃-alkoxy) or —(C₁-C₃-alkyl)-O—(C₁-C₃-alkyl),
and at least one of the substituents R2, R7 and R8 is not H, and
the TEDA derivative (Ib) is a compound of the formula (Ib1) 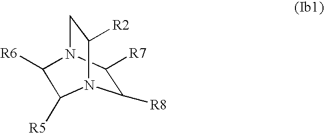
wherein R2, R7 and R8 are each independently H, —OH, —(C₁-C₃-alkyl)-OH, —(C₁-C₃-alkyl)-O—(C₁-C₃-alkyl), —CN, —O-phenyl, —(C₁-C₃-alkyl)-O-phenyl, C₁-C₃-alkoxy, —C(O)(C₁-C₃-alkoxy), —C(O)OH, —N(CH₃)₂, —NH(CH₃), —NH₂, —(C₁-C₃-alkyl)-N(CH₃)₂, —(C₁-C₃-alkyl)-NH(CH₃), —(C₁-C₃-alkyl)-NH₂, or —(C₁-C₃-alkyl)-O—C(O)(C₁-C₃-alkoxy), R5 and R6 are each independently H, C₁-C₃-alkyl, —C(O)OH, —C(O)(C₁-C₃-alkoxy) or —(C₁-C₃-alkyl)-O—(C₁-C₃-alkyl),
and at least one of the substituents R2, R7 and R8 is not H.

15. The process according to claim 11, wherein step a) is carried out in the absence of bases.

16. The process according to claim 11, wherein the dihydropyrazine (II) is prepared by reacting a dicarbonyl compound with ethylenediamine (EDA) or an EDA derivative.

17. The process according to claim 16, wherein the reaction is effected in a moderately polar but water-immiscible solvent, and using equimolar amounts of EDA derivative to give dicarbonyl compound.

18. The process according to claim 16, wherein the reaction is effected in a tert-butyl methyl ether solvent, and using equimolar amounts of EDA derivative to give dicarbonyl compound.

19. A triethylenediamine (TEDA) derivative of the general formula (Ia) or (Ib) 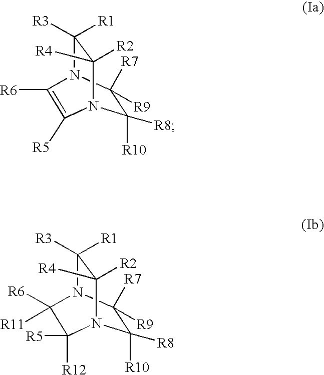
obtainable by the process according to claim 11, where at least one of the substituents to R10 comprises at least one heteroatom selected from halogen, O, P, S and N, or at least one of the R1 to R10 radicals comprises —OH or —NH₂,
with the prerequisite that one of the R1 to R12 radicals is not —C(O)OH, —C(O)OCH₃, —C(O)OC₂H₅, —CH₂—OH, —CH₂—O-benzyl or —CH₂—O—C(O)—CH₃ when the other R1 to R12 radicals are each hydrogen, and two adjacent R1 to R12 radicals are not both —CH₂—O-benzyl when the other R1 to R12 radicals are each hydrogen.

20. A triethylenediamine (TEDA) derivative of the general formula (Ia) or (Ib) 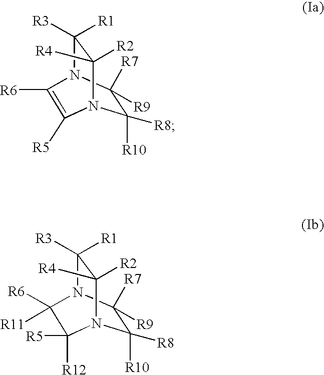
obtainable by the process according to claim 11, where at least one of the substituents R1 to R10 comprises at least one heteroatom selected from O and N, or at least one of the R1 to R10 radicals comprises —OH or —NH₂,
with the prerequisite that one of the R1 to R12 radicals is not —C(O)OH, —C(O)OCH₃, —C(O)OC₂H₅, —CH₂—OH, —CH₂—O-benzyl or —CH₂—O—C(O)—CH₃ when the other R1 to R12 radicals are each hydrogen, and two adjacent R1 to R12 radicals are not both —CH₂—O-benzyl when the other R1 to R12 radicals are each hydrogen.

21. A method for preparing polyurethanes which comprises utilizing the triethylenediamine derivative according to claim 19.

22. A polyurethane comprising at least one triethylenediamine derivative according to claim 19.
